(19) United States
(12) Patent Application Publication
(10) Pub. No.: US 2023/0157877 A1
(43) Pub. Date: May 25, 2023

(54) MULTIPLEXING A LASER BEAM TO FRAGMENT EYE FLOATERS

(71) Applicant: Alcon Inc., Fribourg (CH)

(72) Inventor: Zsolt Bor, San Clemente, CA (US)

(21) Appl. No.: 17/937,974

(22) Filed: Oct. 4, 2022

Related U.S. Application Data
(60) Provisional application No. 63/281,255, filed on Nov. 19, 2021.

(51) Int. Cl. A61F 9/008

(52) U.S. Cl. CPC A61F 9/008; A61B 2017/00154

(57) ABSTRACT

In certain embodiments, an ophthalmic laser system includes a laser device, an ophthalmic microscope, and a controller. The laser device directs laser pulses towards a target within an eye. The target has a dimension. The laser device includes a laser configured to generate a laser beam and one or more laser beam multiplexers. A laser beam multiplexer modulates the laser beam to yield a pulse pattern of laser pulses. The pulse pattern has a coverage related to the dimension of the target to limit movement of the target. The ophthalmic microscope gathers light reflected from within the eye to yield an image of the eye. The controller instructs the laser device to direct the laser pulses towards the target to yield the pulse pattern of laser pulses.

### TECHNICAL FIELD

The present disclosure relates generally to laser vitreolysis systems and methods, and more particularly to multiplexing laser beams to fragment eye floaters.

### BACKGROUND

In ophthalmic laser surgery, a surgeon may direct a laser beam into the eye to treat the eye. For example, a laser beam may be directed into the vitreous to disintegrate eye floaters. Eye floaters are clumps of collagen proteins that form in the vitreous. These clumps disturb vision with moving shadows and distortions. The laser beam may be used to remove the floaters, thus improving vision.

### BRIEF SUMMARY

In certain embodiments, an ophthalmic laser system includes a laser device, an ophthalmic microscope, and a controller. The laser device directs laser pulses towards a target within an eye. An axis of the eye defines a z-axis. The z-axis defines an xy-plane orthogonal to the z-axis, and the xy-plane defines a target xy-plane where the target is located. The target has a dimension in the target xy-plane. The laser device includes a laser configured to generate a laser beam and one or more laser beam multiplexers. Each laser beam multiplexer modulates the laser beam to yield a pulse pattern of laser pulses in the target xy-plane. The pulse pattern has coverage related to the dimension of the target to limit movement of the target. The ophthalmic microscope gathers light reflected from within the eye to yield an image of the eye. The controller instructs the laser device to direct the laser pulses towards the target to yield the pulse pattern of laser pulses

Embodiments may include none, one, some, or all of the following features:
The target is an eye floater.
The multiplexer(s) include a first multiplexer configured to yield a first pulse pattern and a second multiplexer configured to yield a second pulse pattern. The controller may instruct the laser device to use the first multiplexer or the second multiplexer. The first pulse pattern may provide smaller coverage, and the second pulse pattern may provide larger coverage. The first pulse pattern may provide sparser coverage, and the second pulse pattern may provide denser coverage.
The multiplexer(s) include a spatial light modulator that creates a first pulse pattern or a second pulse pattern. The controller may instruct the spatial light modulator to create the first pulse pattern or the second pulse pattern. The first pulse pattern may provide smaller coverage, and the second pulse pattern may provide larger coverage. The first pulse pattern may provide sparser coverage, and the second pulse pattern may provide denser coverage.
The controller determines the dimension of the target from user input.
The controller determines the dimension of the target by performing image processing on the image of the eye to measure the dimension.
The controller selects the pulse pattern of laser pulses in accordance with the dimension of the target.
A laser beam multiplexer may be a diffractive optical element (DOE), a diffraction grating, a holographic optical element (HOE), an interferometer, a spatial light modulator (SLM), a polarization multiplexer, or any combination of the preceding.

In certain embodiments, a method for using an ophthalmic laser system includes instructing, by a controller, a laser device to direct laser pulses towards a target within an eye to yield a pulse pattern of laser pulses. An axis of the eye defines a z-axis. The z-axis defines an xy-plane orthogonal to the z-axis, and the xy-plane defines a target xy-plane where the target is located. The target has a dimension in the target xy-plane. A laser beam is generated by a laser of the laser device. The laser beam is modulated, by laser beam multiplexer(s) of the laser device, to yield the pulse pattern. The pulse pattern has coverage related to the dimension of the target to limit movement of the target. The laser pulses are directed, by the laser device, towards the target within the eye. Light reflected from within the eye is gathered, by an ophthalmic microscope, to yield an image of the eye.

Embodiments may include none, one, some, or all of the following features:
The multiplexer(s) include a first multiplexer configured to yield a first pulse pattern and a second multiplexer configured to yield a second pulse pattern. The method may include instructing, by the controller, the laser device to use the first multiplexer or the second multiplexer.
The multiplexer(s) include a spatial light modulator that creates a first pulse pattern or a second pulse pattern. The method may include instructing, by the controller, the spatial light modulator to create the first pulse pattern or the second pulse pattern.

In certain embodiments, an ophthalmic laser system includes a laser device, an ophthalmic microscope, and a controller. The laser device directs laser pulses towards a target within an eye, where the target is an eye floater. An axis of the eye defines a z-axis. The z-axis defines an xy-plane orthogonal to the z-axis, and the xy-plane defines a target xy-plane where the target is located. The target has a dimension in the target xy-plane. The laser device includes a laser configured to generate a laser beam and one or more laser beam multiplexers. Each laser beam multiplexer modulates the laser beam to yield a pulse pattern of laser pulses in the target xy-plane. The pulse pattern has a coverage related to the dimension of the target to limit movement of the target. The multiplexer(s) includes a first multiplexer configured to yield a first pulse pattern and a second multiplexer configured to yield a second pulse pattern, or a spatial light modulator configured to create the first pulse pattern and the second pulse pattern. The first pulse pattern provides smaller coverage, and the second pulse pattern provides larger coverage. The first pulse pattern provides sparser coverage, and the second pulse pattern provides denser coverage. A laser beam multiplexer may be a diffractive optical element (DOE), a diffraction grating, a holographic optical element (HOE), an interferometer, a spatial light modulator (SLM), a polarization multiplexer, or any combination of the preceding. The ophthalmic microscope gathers light reflected from within the eye to yield an image of the eye. The controller instructs the laser device to direct the laser pulses towards the target to yield the pulse pattern of laser pulses. The controller instructs the laser device to direct the plurality of laser pulses towards the target to yield the pulse pattern of laser pulses.

Embodiments may include the following feature:

The controller: determines the dimension of the target from user input or by performing image processing on the image of the eye to measure the dimension; selects the pulse pattern of laser pulses in accordance with the dimension of the target; and instructs the laser device to use the first multiplexer or the second multiplexer or instructs the spatial light modulator to create the first pulse pattern or the second pulse pattern.

### BRIEF DESCRIPTION OF THE DRAWINGS

FIG. 1 illustrates an example of an ophthalmic laser system that may be used to perform laser vitreolysis on a patient eye, according to certain embodiments;

FIG. 2 illustrates an example of a laser pulse causing a floater to jump;

FIG. 3 illustrates an example of the coverage of a pulse pattern relative to a floater;

FIG. 4 illustrates an example of a multiplexed pattern, which may be used by the ophthalmic laser system of FIG. 1; and

FIGS. 5A to 5D illustrate examples of multiplexed patterns, which may be used by the ophthalmic laser system of FIG. 1; and

FIG. 6 illustrates an example of a method for fragmenting eye floaters, which may be used by the ophthalmic laser system of FIG. 1.

### DESCRIPTION OF EXAMPLE EMBODIMENTS

Referring now to the description and drawings, example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

In laser vitreolysis, laser pulses are used to disintegrate eye floaters to improve vision. In certain situations, the laser pulses have a pulse energy of approximately 3 to 10 milliJoules (mJ), which can create a rapidly expanding cavitation bubble. The acceleration of the bubble-vitreous interface can reach a point where it can mechanically disintegrate a floater. If the pulse hits the center of a floater, the bubble disintegrates the floater. However, if the pulse hits the periphery, the bubble rapidly pushes the floater, causing it to jump.

In certain embodiments, a laser device directs laser pulses towards a floater within an eye. The laser device includes a laser beam multiplexer that splits a laser beam to yield a pattern of pulses where some of the pulses surround the floater, reducing the likelihood the floater will jump.

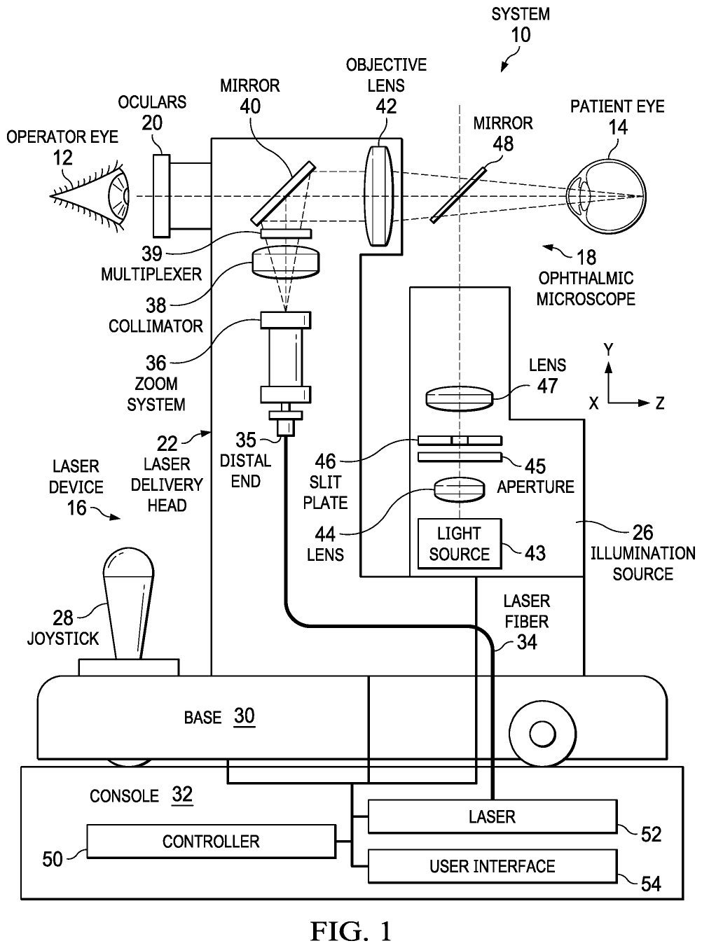

FIG. 1 illustrates an example of an ophthalmic laser system 10 that an operator (with an operator eye 12) may use to perform laser vitreolysis on a patient eye 14 to remove vitreous floaters, according to certain embodiments. Vitreous floaters are microscopic collagen fibers within the vitreous that tend to clump together. These clumps scatter light and cast shadows on the retina, which appear as visual disturbances in the vision of the patient. Ophthalmic laser system 10 allows the operator to see floaters in relation to the retina and lens of the eye, and then direct a laser beam to break up the floaters. In the illustrated example, patient eye 14 has an axis (visual or optical) that defines a z-axis. The z-axis defines an x-axis and a y-axis orthogonal to the z-axis. In turn, the x-axis and the y-axis define an xy-plane.

In the example, ophthalmic laser system 10 comprises oculars 20, a laser delivery head 22, a slit illumination source 26, a positioning device (such as a joystick 28), a base 30, and a console 32, coupled as shown. Laser delivery head 22 includes a laser fiber 34, a distal end 35, a zoom system 36, a collimator 38, a beam multiplexer 39, a mirror 40, and an objective lens 42, coupled as shown. Slit illumination source 26 includes a light source 43, condenser lens 44, a variable aperture 45, a variable slit plate 46, a projection lens 47, and a mirror 48. Console 32 includes a computer (such as a controller 50), a laser 52, and a user interface 54, coupled as shown.

As an overview, ophthalmic laser system 10 includes a laser device 16 (e.g., laser 52, laser fiber 34, and laser delivery head 22) and an ophthalmic microscope 18 such as a slit lamp (e.g., oculars 20, objective lens 42, mirror 48, and slit illumination source 26). Operator eye 12 utilizes the optical path from oculars 20 through mirror 40, objective lens 42, and mirror 48 to view patient eye 14. A laser beam follows the laser path from laser 52 through laser delivery head 22 and mirror 48 to treat patient eye 14.

According to the overview, laser device 16 directs a laser beam comprising laser pulses towards a target within eye 14. The target has a dimension (e.g., length) in the xy-plane where the target is located. Ophthalmic microscope 18 gathers light reflected from within eye 14 to yield an image of eye 14. Beam multiplexer 39 splits the laser beam into a plurality of laser beams or otherwise modulates the laser beam to yield a plurality of laser pulses. Controller 50 instructs laser device 16 to direct the laser pulses towards the target such that a subset of the laser pulses surround the target, reducing the likelihood of causing the target to jump.

In more detail, in certain embodiments, oculars 20 allow operator eye 12 to view patient eye 14. Laser delivery head 22 delivers a laser beam of laser pulses from laser 52 of console 32 to patient eye 14. Laser fiber 34 of delivery head 22 transports the laser beam from laser 52 to the end of fiber 34. Zoom system 36 includes optical elements that change the spot size of the laser beam that exits fiber 34. Collimator 38 collimates the laser beam, and mirror 40 directs the beam through objective lens 42, which focuses the beam. Zoom system 36 and collimator 38 are configured to direct a parallel laser beam to mirror 40, in order to focus the laser beam onto the image plane of ophthalmic microscope 18. Mirror 40 may be a dichroic mirror that is reflective for the laser beam wavelength and transmissive for visible light.

Slit illumination source 26 of laser system 10 provides light that illuminates the surgical site of patient eye 14. In certain embodiments, slit illumination source 26 may illuminate a floater coaxially with the laser beam or at an oblique angle to the beam. Such oblique illumination reduces light scattered from the cornea and human lens and also reduces red reflex from the retina. Oblique illumination resembles dark field illumination.

Slit illumination source 26 includes light source 43, which emits light such as a high-intensity illumination light. Condenser lens 44 directs the light towards variable aperture 45 and variable slit plate 46. Variable aperture 45 defines the height of the light in the y-direction, and variable slit plate 43 defines the width of the light in the x-direction to form the light into a slit shape. Projection lens 47 directions the light towards prism mirror 48, which directs the slit of light into patient eye 14.

Base 30 supports laser delivery head 22 and slit illumination source 26. Joystick 28 moves base 30 in the x-, y-, and z-directions. Console 32 includes components that support the operation of system 10. Controller 50 of console 32 controls of the operation of components of system 10, e.g., base 30, laser delivery head 22, slit illumination source 26, laser 52, and/or user interface 54. Laser 52 supplies the laser beam. Any suitable laser 30 may be used, e.g., a femtosecond or nanosecond laser (e.g., Q-switched) with any suitable crystal (e.g., Nd:YAG, Erbium:YAG, Ti: Sapphire, or ruby). The laser beam may have any suitable wavelength, e.g., in a range from 500 nm to 1100 nm. User interface 54 communicates information between the operator and system 10.

Laser beam multiplexer 39 multiplexes (e.g., splits or otherwise modulates) a laser beam to form a plurality of laser pulses that yield a multiplexed focal pulse pattern. A multiplexed pulse pattern distributes pulses in the x-, y-, and/or z-directions. For example, the pulses may form a pattern, e.g., an array, in the xy-plane of the floater. As another example, the pulses may form multiple patterns (e.g., arrays) in the z-direction and parallel to the xy-plane of the floater, yielding a three-dimensional (3D) volume (e.g., a 3D array).

Laser beam multiplexer 39 comprises any suitable optical element that can split a laser beam into more than one beam or otherwise modulate the laser beam to yield a pulse pattern with two or more pulses. In general, an optical element is a component that can act on (e.g., transmit, reflect, refract, diffract, collimate, condition, shape, focus, modulate, and/or otherwise act on) a laser beam. Examples of laser beam multiplexer 39 include a diffractive optical element (DOE), a diffraction grating, a holographic optical element (HOE), an interferometer (e.g., a Michelson, Mach-Zehnder, or other interferometer), a spatial light modulator (SLM), a polarization multiplexer (e.g., Wollaston prism), and a combination of different beam multiplexers (e.g., 5× diffractive multiplexer and a Wollaston-doubler).

Laser device 16 may include one or more multiplexers 39 that yield one or more pulse patterns. In certain embodiments, laser device 16 is a simple device that includes one multiplexer 39 that yields one pulse pattern. In other embodiments, laser device 16 includes a plurality of multiplexers that yield different pulse patterns. Each multiplexer can be moved into and out of the beam path with a mechanical actuator. In the embodiments, in response to the selection of a pattern by, e.g., a surgeon, controller 50 identifies the multiplexer 39 that yields the selected pattern, and instructs an actuator to move the identified multiplexer 39 into place. In response, the actuator moves the identified multiplexer 39 into the laser beam path.

In yet other embodiments, the multiplexer 39 is a spatial light modulator (SLM) that can modulate amplitude, phase, and/or polarization of the laser beam in space and/or time to produce different patterns. In the embodiments, in response to the selection of a pattern by, e.g., a surgeon, controller 50 instructs the spatial light modulator to modulate the laser beam to produce the selected pattern.

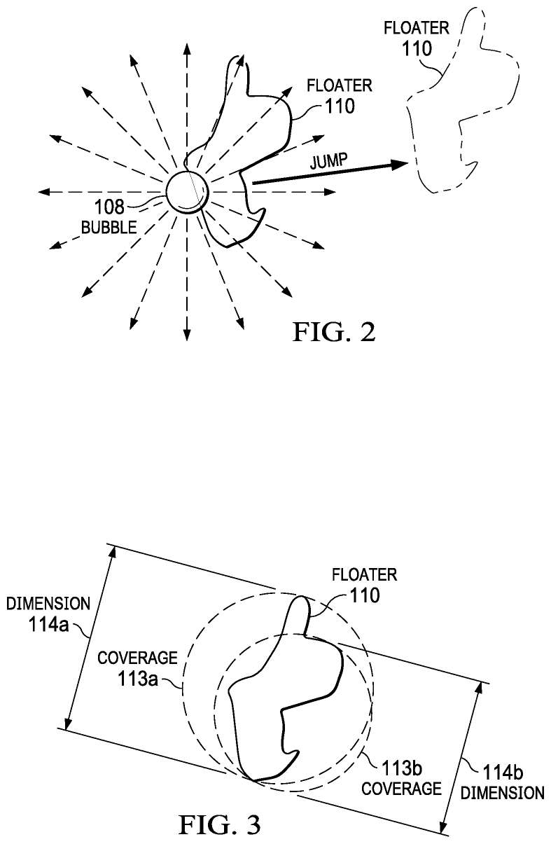

FIG. 2 illustrates an example of a laser pulse causing a floater 110 to jump. In the example, floater 110 is approximately 100 to 300 microns across. A 1 milliJoule (mJ) laser pulse creates a rapidly expanding cavitation bubble 108 with a peak diameter of approximately 1 millimeter (mm) in approximately 1.19 milliseconds (ms). If the pulse hits the center of floater 110, the bubble fragments floater 110. However, if the pulse hits the periphery of floater 110, the bubble rapidly pushes floater 110, causing it to jump. In the example, floater 110 moves a distance of, e.g., 1 mm, such that the laser will have to be redirected with the positioning device.

FIG. 3 illustrates an example of the coverage 113 of a pulse pattern 112 relative to a floater 110. In the illustrated example, the dashed lines represent the pulse coverage 113 of a pulse pattern 112, i.e., the area enclosed by the outermost pulses of the pulse pattern. Pulse pattern 112 places a subset (which may be part of a set or the whole set) of the pulses in the path where floater 110 could jump in order to limit the movement of floater 110. Accordingly, coverage 113 of pulse pattern 112 may be larger than at least a majority of floater 110. For example, coverage 113 may be at least as large as, or at least as 25, 40, or 50 percent larger than floater 110. Floaters 110 tend to move more in the x- and y-directions than in the z-direction, so pulse pattern 112 may place more pulses in the x- and y-directions around floater 110.

The coverage 113 may be at least as large as a dimension 114 that indicates the general size of floater 110 such that the outermost portion of coverage 113 surrounds dimension 114. Dimension 114 may be measured in any suitable direction in three-dimensional space. In certain embodiments, ophthalmic microscope 18 provides an image of floater 110 in a target xy-plane where floater 110 (e.g., approximately the centroid of floater 110) is located, so dimension 114 is measured in the target xy-plane.

Dimension 114 (114a, 114b) may measure any suitable portion of floater 110 that indicates the size of floater 110. For example, dimension 114a measures the longest part of floater 110, and dimension 114b measures the longest part of a majority (e.g., 50 to 70, 70 to 90, and/or 90 to 100 percent) of the area of floater 114b. In some cases, dimension 114b may be used to provide greater coverage if using dimension 114a still causes floaters 110 to jump. A pulse pattern 112 with coverage 113 (113a, 113b) may be selected according to dimension 114. For example, coverage 113a covers dimension 114a, and coverage 113b covers dimension 114b. Coverage 113 may be substantially centered about the centroid of floater 110 to reduce the likelihood of jumping.

Laser device 16 may include one or more multiplexers 39 that yield patterns with different coverage 113, e.g., one multiplexer provides smaller coverage and another provides larger coverage. For example, laser device 16 includes multiple multiplexers 39 that yield patterns with different coverage 113, or one multiplexer 39 (e.g., a SLM) that can create patterns with different coverage 113. The coverage 113 may be for smaller (e.g., 50 μm) to larger (e.g., 1 mm) floaters, e.g., a range of 10 microns to 5 mm, such as 20 microns to 3 mm. Coverage 113 may be divided into ranges (which may overlap), where a particular pattern yields a particular range. For example, coverage 113 is divided into smaller coverage for smaller floaters 110 (e.g., 20 microns to 120 microns), average coverage for the most common size of floaters 110 (e.g., 100 microns to 1 mm), and larger coverage for larger floaters 110 (e.g., 0.9 to 3 mm). In the example, one pattern provides smaller floater coverage, another provides average floater coverage, and yet another provides larger floater coverage.

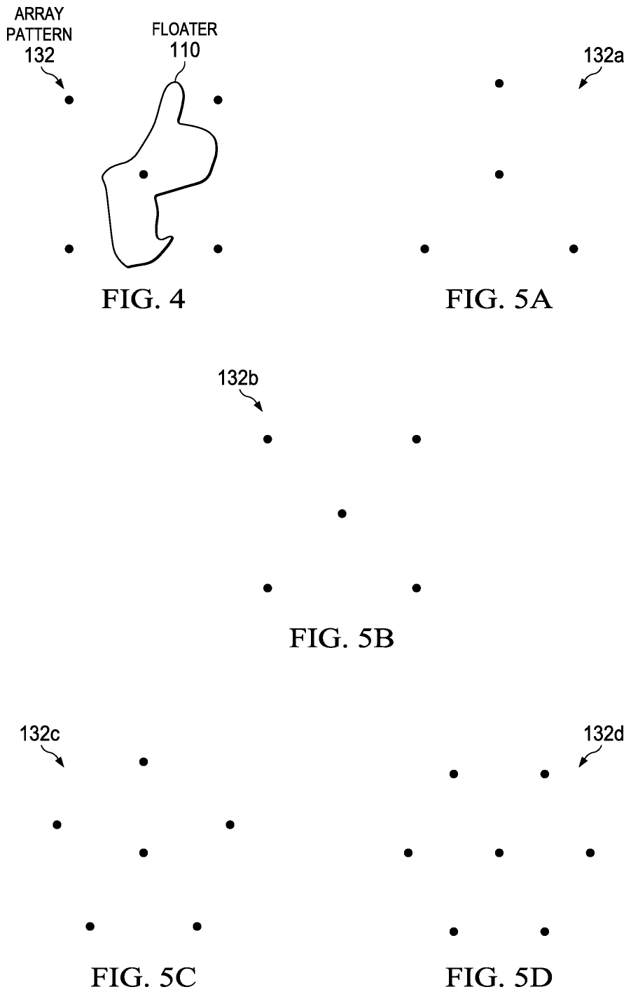

FIG. 4 illustrates an example of a multiplexed array pattern 132 in the xy-plane, which may be used by ophthalmic laser system 10 of FIG. 1. A laser beam is multiplexed (e.g., split or otherwise modulated) into multiple beams (e.g., 2 to 5, 6 to 9, or 10 or more beams) to form laser pulses that surround floater 110. In the illustrated example, the pulse coverage of pattern 132 is at least as large as the longest part of a majority (approximately 90%) of floater 110. The outermost pulses surround the majority of floater 110, and the central pulse hits floater 110. In other examples, the coverage of the array is at least as large as the longest dimension of floater 110, such that the outermost pulses of the array surround the whole floater 110.

FIGS. 5A to 5D illustrate examples of multiplexed array patterns 132 (132a to 132d) in the xy-plane, which may be used by ophthalmic laser system 10 of FIG. 1. A pulse pattern may have any suitable pattern in the xy-plane of the target. In the examples, patterns 132 are symmetrical about a central pulse. Pattern 132a includes a central pulse with three outer pulses that form a triangle. Pattern 132b includes a central pulse with four outer pulses that form a square. Pattern 132c includes a central pulse with five outer pulses that form a pentagram. Pattern 132d includes a central pulse with six outer pulses that form a hexagon.

In the examples, patterns 132 have different pulse densities. Pattern 132a has the sparsest coverage, pattern 132b has denser coverage, pattern 132c has even denser coverage, and pattern 132d has the densest coverage. A pattern 132 with sparser coverage may be used for thinner, sparser floaters, and a pattern 132 with a denser coverage may be used for thicker, denser floaters.

Similar to multiplexers 39 discussed above with patterns of different coverage, laser device 16 may include one or more multiplexers 39 that yield patterns of different density. For example, laser device 16 includes multiple multiplexers 39 that yield patterns of different density or one multiplexer 39 (e.g., a SLM) that can create patterns of different density.

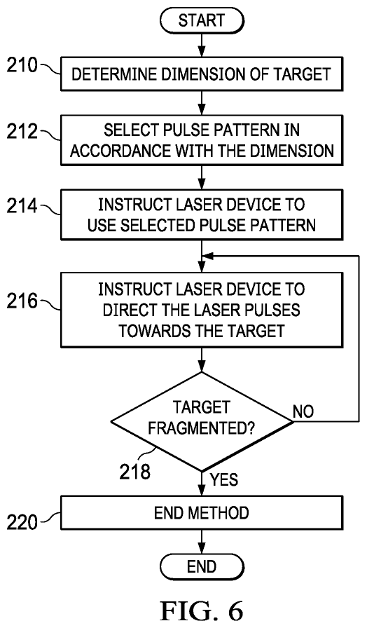

FIG. 6 illustrates an example of a method for fragmenting eye floaters, which may be used by ophthalmic laser system 10 of FIG. 1. In the example, controller 50 of system 10 may perform at least some steps of the method. The method starts at step 210, where a dimension of the target, e.g., floater 110, is determined. In certain embodiments, the user determines the dimension of the target. In other embodiments, controller 50 determines the dimension of the target by receiving user input of the dimension. In yet other embodiments, controller 50 performs image processing on an image of the target (provided by, e.g., the ophthalmic microscope) to measure the dimension.

A pulse pattern is selected in accordance with the dimension at step 212. As described above, when treating floaters, coverage larger than most of the area of the floater reduces the likelihood that the floater will jump and increases the likelihood of disintegrating the floater. In certain embodiments, the user selects the pulse pattern. In other embodiments, controller 50 selects the pulse pattern in response to user input of the selection. In yet other embodiments, controller 50 automatically selects a pulse pattern that covers the dimension of the target.

Controller 50 instructs laser device 16 to use the selected pattern at step 214. In certain embodiments, controller 50 instructs the laser device to use a laser beam multiplexer that yields the selected pulse pattern. In other embodiments, controller 50 instructs a spatial light modulator to create the selected pulse pattern.

Controller 50 instructs laser device 16 to direct the laser pulses towards the target at step 216. The user (e.g., using joystick 28) or controller 50 (e.g., using tracking) may aim the pulses. Aiming the pulses such that the approximate center of the pattern hits the approximate center of the target decreases the likelihood that the target jumps.

The target may be fragmented at step 218. If the target has not been fragmented, the method returns to step 216 where controller 50 instructs laser device 16 to direct the laser beam towards the target. The laser beam may need to be re-aimed prior to directing the laser beam. If the target has been fragmented, the method proceeds to step 220 to end the method. The method then ends.

A component (such as controller 50) of the systems and apparatuses disclosed herein may include an interface, logic, and/or memory, any of which may include computer hardware and/or software. An interface can receive input to the component and/or send output from the component, and is typically used to exchange information between, e.g., software, hardware, peripheral devices, users, and combinations of these. A user interface is a type of interface that a user can utilize to communicate with (e.g., send input to and/or receive output from) a computer. Examples of user interfaces include a display, Graphical User Interface (GUI), touchscreen, keyboard, mouse, gesture sensor, microphone, and speakers.

Logic can perform operations of the component. Logic may include one or more electronic devices that process data, e.g., execute instructions to generate output from input. Examples of such an electronic device include a computer, processor, microprocessor (e.g., a Central Processing Unit (CPU)), and computer chip. Logic may include computer software that encodes instructions capable of being executed by an electronic device to perform operations. Examples of computer software include a computer program, application, and operating system.

A memory can store information and may comprise tangible, computer-readable, and/or computer-executable storage medium. Examples of memory include computer memory (e.g., Random Access Memory (RAM) or Read Only Memory (ROM)), mass storage media (e.g., a hard disk), removable storage media (e.g., a Compact Disk (CD) or Digital Video or Versatile Disk (DVD)), database, network storage (e.g., a server), and/or other computer-readable media. Particular embodiments may be directed to memory encoded with computer software.

Although this disclosure has been described in terms of certain embodiments, modifications (such as changes, substitutions, additions, omissions, and/or other modifications) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, or the operations of the systems and apparatuses may be performed by more, fewer, or other components, as apparent to those skilled in the art. As another example, modifications may be made to the methods disclosed herein. The methods may include more, fewer, or other steps, and the steps may be performed in any suitable order, as apparent to those skilled in the art.

To aid the Patent Office and readers in interpreting the claims, Applicants note that they do not intend any of the claims or claim elements to invoke 35 U.S.C. § 112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the applicants to refer to structures known to those skilled in the relevant art and is not intended to invoke 35 U.S.C. § 112(f).

What is claimed:

1. An ophthalmic laser system, comprising:
a laser device configured to direct a plurality of laser pulses towards a target within an eye, an axis of the eye defining a z-axis, the z-axis defining an xy-plane orthogonal to the z-axis, the xy-plane defining a target xy-plane where the target is located, the target having a dimension in the target xy-plane, the laser device comprising:
a laser configured to generate a laser beam; and
one or more laser beam multiplexers, each laser beam multiplexer configured to modulate the laser beam to yield a pulse pattern of laser pulses in the target xy-plane, the pulse pattern having a coverage related to the dimension of the target to limit movement of the target;
an ophthalmic microscope configured to gather light reflected from within the eye to yield an image of the eye; and
a controller configured to:
instruct the laser device to direct the plurality of laser pulses towards the target to yield the pulse pattern of laser pulses.

2. The ophthalmic laser system of claim 1, the target comprising an eye floater.

3. The ophthalmic laser system of claim 1, the one or more laser beam multiplexers comprising:
a first multiplexer configured to yield a first pulse pattern; and
a second multiplexer configured to yield a second pulse pattern.

4. The ophthalmic laser system of claim 3, the controller further configured to:
instruct the laser device to use the first multiplexer or the second multiplexer.

5. The ophthalmic laser system of claim 3:
the first pulse pattern providing smaller coverage; and
the second pulse pattern providing larger coverage.

6. The ophthalmic laser system of claim 3:
the first pulse pattern providing sparser coverage; and
the second pulse pattern providing denser coverage.

7. The ophthalmic laser system of claim 1, the one or more laser beam multiplexers comprising a spatial light modulator configured to:
create a first pulse pattern; and
create a second pulse pattern.

8. The ophthalmic laser system of claim 7, the controller further configured to:
instruct the spatial light modulator to create the first pulse pattern or the second pulse pattern.

9. The ophthalmic laser system of claim 7:
the first pulse pattern providing smaller coverage; and
the second pulse pattern providing larger coverage.

10. The ophthalmic laser system of claim 7:
the first pulse pattern providing sparser coverage; and
the second pulse pattern providing denser coverage.

11. The ophthalmic laser system of claim 1, the controller further configured to determine the dimension of the target from user input.

12. The ophthalmic laser system of claim 1, the controller further configured to determine the dimension of the target by performing image processing on the image of the eye to measure the dimension.

13. The ophthalmic laser system of claim 1, the controller further configured to:
select the pulse pattern of laser pulses in accordance with the dimension of the target.

14. The ophthalmic laser system of claim 1, a laser beam multiplexer of the one or more laser beam multiplexers comprising one of the following: a diffractive optical element (DOE), a diffraction grating, a holographic optical element (HOE), an interferometer, a spatial light modulator (SLM), a polarization multiplexer, or any combination of the preceding.

15. A method for using an ophthalmic laser system, comprising:
instructing, by a controller, a laser device to direct a plurality of laser pulses towards a target within an eye to yield a pulse pattern of laser pulses, an axis of the eye defining a z-axis, the z-axis defining an xy-plane orthogonal to the z-axis, the xy-plane defining a target xy-plane where the target is located, the target having a dimension in the target xy-plane;
generating, by a laser of the laser device, a laser beam;
modulating, by one or more laser beam multiplexers of the laser device, the laser beam to yield the pulse pattern, the pulse pattern having a coverage related to the dimension of the target to limit movement of the target;
directing, by the laser device, the laser pulses towards the target within the eye; and
gathering, by an ophthalmic microscope, light reflected from within the eye to yield an image of the eye.

16. The method of claim 15, the one or more laser beam multiplexers comprising:
a first multiplexer configured to yield a first pulse pattern; and
a second multiplexer configured to yield a second pulse pattern.

17. The method of claim 16, further comprising:
instructing, by the controller, the laser device to use the first multiplexer or the second multiplexer.

18. The method of claim 15, the one or more laser beam multiplexers comprising a spatial light modulator configured to:
create a first pulse pattern; and
create a second pulse pattern.

19. The method of claim 18, further comprising:
instructing, by the controller, the laser device to create the first pulse pattern or the second pulse pattern.

20. An ophthalmic laser system, comprising:
a laser device configured to direct a plurality of laser pulses towards a target within an eye, the target comprising an eye floater, an axis of the eye defining a z-axis, the z-axis defining an xy-plane orthogonal to the z-axis, the xy-plane defining a target xy-plane where the target is located, the target having a dimension in the target xy-plane, the laser device comprising:
a laser configured to generate a laser beam; and
one or more laser beam multiplexers, each laser beam multiplexer configured to modulate the laser beam to yield a pulse pattern of laser pulses in the target xy-plane, the pulse pattern having a coverage related to the dimension of the target to limit movement of the target, the one or more laser beam multiplexers comprising a first multiplexer configured to yield a first pulse pattern and a second multiplexer configured to yield a second pulse pattern, or a spatial light modulator configured to create the first pulse pattern or the second pulse pattern, the first pulse pattern providing smaller coverage and the second pulse pattern providing larger coverage, the first pulse pattern providing sparser coverage and the second pulse pattern providing denser coverage, a laser beam multiplexer of the one or more laser beam multiplexers comprising one of the following: a diffractive optical element (DOE), a diffraction grating, a holographic optical element (HOE), an interferometer, a spatial light modulator (SLM), a polarization multiplexer, or any combination of the preceding;
an ophthalmic microscope configured to gather light reflected from within the eye to yield an image of the eye; and
a controller configured to:
instruct the laser device to direct the plurality of laser pulses towards the target to yield the pulse pattern of laser pulses.

21. The ophthalmic laser system of claim 20, the controller further configured to:
determine the dimension of the target from user input or by performing image processing on the image of the eye to measure the dimension;
select the pulse pattern of laser pulses in accordance with the dimension of the target; and
instruct the laser device to use the first multiplexer or the second multiplexer or instruct the spatial light modulator to create the first pulse pattern or the second pulse pattern.
